(19) United States
(12) Patent Application Publication
(10) Pub. No.: US 2020/0281681 A1
(43) Pub. Date: Sep. 10, 2020

(54) PARKING SURGICAL INSTRUMENTS WITHIN A STERILE FIELD USING FORCE ATTENUATING POLYMER GEL

(71) Applicant: SG, LLC, Boston, MA (US)

(72) Inventor: Paul Andrew Glazer, Boston, MA (US)

(21) Appl. No.: 16/811,711

(22) Filed: Mar. 6, 2020

Related U.S. Application Data
(60) Provisional application No. 62/815,043, filed on Mar. 7, 2019.

(51) Int. Cl. A61B 50/33; A61B 46/23; A61L 31/04

(52) U.S. Cl. CPC A61B 50/33; A61B 2050/002; A61L 31/04; A61B 46/23

(57) ABSTRACT

A system for parking and maintaining surgical instruments within a sterile field includes, a layer of force attenuating polymer gel configured and adapted to be sterilized and attached to a structure within the sterile field and at least one or more of: a sterile surgical tray and a sterile surgical drape.

### CROSS-REFERENCE TO RELATED APPLICATIONS

This Application claims the benefit of U.S. Provisional Patent Application No. 62/815,043 filed Mar. 7, 2019, which is incorporated by reference herein in its entirety.

### BACKGROUND OF THE INVENTION

### 1. Field of the Invention

The invention generally relates to polymeric compounds and in particular to a viscoelastic, force-attenuating elastomeric compound with adhesive properties and to its application to improved position maintenance for parked surgical instruments in a sterile field during a surgical procedure.

### 2. Description of the Related Art

When surgical instruments are maintained with only the force of gravity they can often fall out of the sterile field during a procedure, requiring a new instrument to be opened or the current instrument to be sterilized. This can lead to extended duration of the procedure, and increased cost of the procedure. Additionally, surgical instruments are often required to be securely parked in a location that is inconvenient for the procedure.

Such conventional methods and systems have generally been considered satisfactory for their intended purposes. However, there is a need in the art for improved position maintenance of surgical instruments during surgical procedures. The present invention provides a solution for these needs.

### SUMMARY OF THE INVENTION

A system for parking and maintaining surgical instruments within a sterile field includes a layer of force attenuating polymer gel configured and adapted to be sterilized and attached to a structure within the sterile field.

The layer force attenuating polymer gel can be sterilized and attached as a tray liner to a surgical tray for parking surgical instruments within the sterile field and the layer of force attenuating polymer gel and the surgical tray can be sterilized and enclosed in a sterile package.

The layer of force attenuating polymer gel can be sterilized and can be sewn in to a surgical drape for parking surgical instruments within the sterile field. The layer of force attenuating polymer gel can be attached to a surgical drape in a manner where the parked surgical instruments are maintained at an angle that is less than forty-five degrees relative to the horizontal plane and wherein the surgical drape and sewn in layer of force attenuating polymer gel can be sterilized and are enclosed in a sterile package.

At least one piece of two sided adhesive tape can be attached on one side to the layer of force attenuating polymer gel for attaching to a surgical drape and wherein the layer of force attenuating polymer gel with attached adhesive tape can be sterilized and enclosed in a sterile package. At least one piece of a hook and loop fastener can be attached on one side to the layer of force attenuating polymer gel for attaching to a surgical drape. One portion of the hook and loop fastener is attached to the layer of force attenuating polymer gel and the opposite portion of the hook and loop fastener is attached to the surgical drape. The portion of the hook and loop fastener attached to the surgical drape can be sewn into the surgical drape or, the portion of the hook and loop fastener that can be attached to the surgical drape is free to be placed anywhere along the surgical drape. The layer of force attenuating polymer gel with attached hook and loop fastener can be sterilized and enclosed in a sterile package. At least one alligator clip can be attached to the layer of force attenuating polymer gel for attaching to a surgical drape and wherein the layer of force attenuating polymer gel with attached alligator clips can be sterilized and enclosed in a sterile package.

These and other features of the systems and methods of the subject disclosure will become more readily apparent to those skilled in the art from the following detailed description of the preferred embodiments taken in conjunction with the drawings.

### BRIEF DESCRIPTION OF THE DRAWINGS

So that those skilled in the art to which the subject invention appertains will readily understand how to make and use the devices and methods of the subject inventions without undue experimentation, preferred embodiments thereof will be described in detail herein below with reference to certain figures wherein:

FIG. 1A is a perspective view of a surgical tray in a sterile field constructed in accordance with embodiments of the present invention, showing a layer of force attenuating polymer gel attached to a surgical tray in a sterile field;

FIG. 1B is a perspective view of the surgical tray of FIG. 1A with an attached layer of force attenuating polymer gel enclosed in a sterile packaging;

FIG. 2A is a perspective view of a surgical drape in a sterile field constructed in accordance with embodiments of the present invention, showing a layer of force attenuating polymer gel sewn into the surgical drape;

FIG. 2B is a perspective view of the surgical drape of FIG. 2A with a sewn in layer of force attenuating polymer gel enclosed in a sterile packaging;

FIG. 3A is a perspective view of a surgical drape in a sterile field constructed in accordance with embodiments of the present invention, showing a layer of force attenuating polymer gel attached to the surgical drape using at least one piece of two sided adhesive tape;

FIG. 3B is a perspective view of a layer of force attenuating polymer gel of FIG. 3A with at least one piece of two sided tape attached and enclosed in a sterile packaging;

FIG. 4A is an exploded view of a surgical drape in a sterile field constructed in accordance with embodiments of the present invention, showing a layer of force attenuating polymer gel attached to the surgical drape using at least one piece of hook and loop fastener;

FIG. 4B is a perspective view of a layer of force attenuating polymer gel of FIG. 4A with at least one piece of hook and loop attached and enclosed in a sterile packaging;

FIG. 5A is a perspective view of a surgical drape in a sterile field constructed in accordance with embodiments of the present invention, showing a layer of force attenuating polymer gel attached to the surgical drape using at least one alligator clip; and

FIG. 5B is a perspective view of a layer of force attenuating polymer gel of FIG. 5A with at least one alligator clip attached and enclosed in a sterile packaging.

### DETAILED DESCRIPTION OF PREFERRED EMBODIMENTS

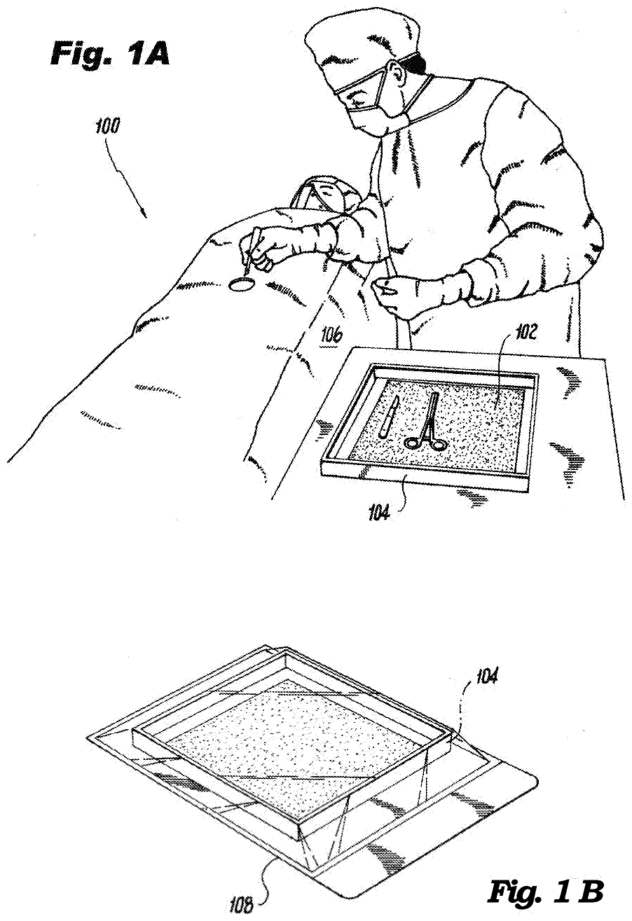

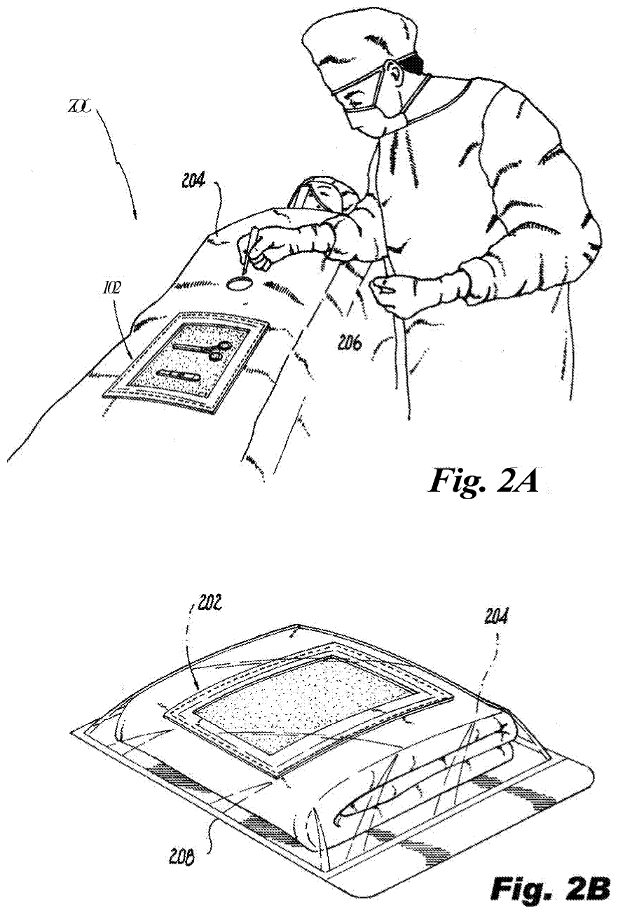

Reference will now be made to the drawings wherein like reference numerals identify similar structural features or aspects of the subject invention. For purposes of explanation and illustration, and not limitation, a perspective view of an exemplary embodiment of a system for parking and maintaining surgical instruments in a sterile field constructed in accordance with embodiments of the invention shown in FIG. 1A and is designated generally by reference character 100. Other embodiments in accordance with the invention, or aspects thereof, are provided in FIGS. 2A-5B as will be described.

As shown in FIG. 1A, system 100 for parking and maintaining surgical instruments in a sterile field 106 includes, a layer 102 of force attenuating polymer gel, referred to as “the gel” or “gel” throughout this description. Also shown is a surgical tray 104 where the layer of gel 102 is attached to the surgical tray 104 for parking surgical instruments when not in use but are maintained within sterile field 106. It is contemplated that the gel can be the gel described in U.S. Pat. No. 7,041,719, the contents of which are hereby incorporated in their entirety. The gel 102 has a tacky outer surface and can be placed on a surgical tray 104 so that when surgical instruments are parked on the gel 102, they are maintained within sterile field 106 even if bumped or tilted. With reference now to FIG. 1B, it is contemplated that the gel 102 and the surgical tray 104 can be sterilized and enclosed in a sterile package 108.

As shown in FIG. 2A, a surgical drape 204 incorporates a layer of the gel 202. The gel layer 202 is sewn into surgical drape 204 in a manner such that when surgical drape 204 is placed over a patient, the gel layer 202 is maintained at an angle less than forty-five degrees relative to the horizontal plane. The tacky nature of the gel 202 will maintain parked surgical instruments within sterile field 204, but will also maintain parked surgical instruments in close proximity to the surgical procedure. It is contemplated that a layer of gel 202 can be incorporated into the surgical drape to reduce the risk of removing surgical instruments from sterile field 206 by maintaining parked surgical instruments in very close proximity to the surgical procedure. As seen in FIG. 2B, it is contemplated that the gel 202 as sewn into surgical drape 204 can be sterilized and enclosed in a sterile package 208.

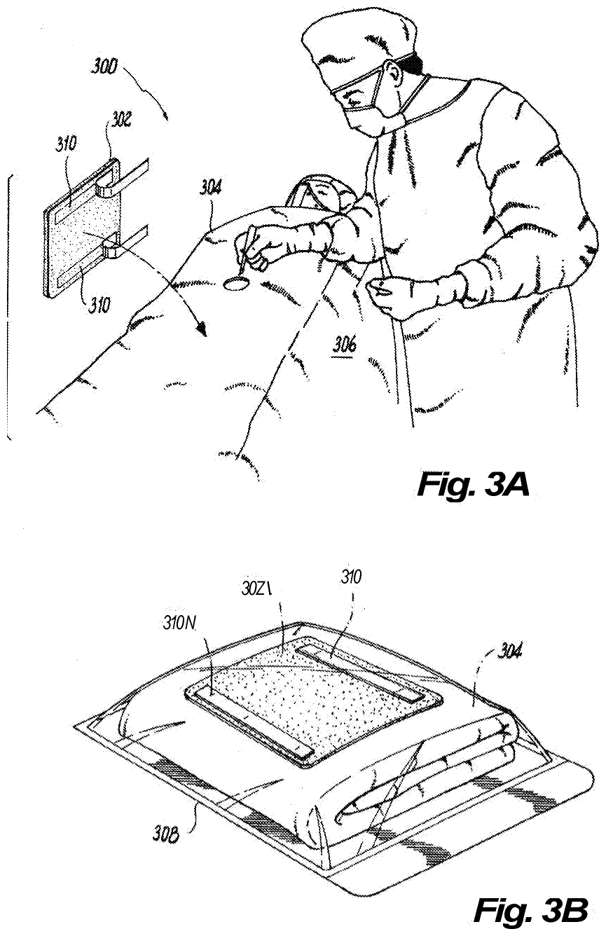

With reference now to FIG. 3A, at least one piece of two sided adhesive tape 310 is affixed to one side of the gel layer 302 so that the gel layer 302 can be attached to the surgical drape 304 by removing the paper backing from the adhesive tape 310 and placing the gel layer 302 onto the surgical drape 304. It is contemplated that the gel 302 and attached adhesive tape can be sterilized and enclosed in a sterile package 308 to maintain the sterile state until needed for use in a surgery. Those skilled in the art would readily appreciate that adhesive tape 310 and surgical drape 304 need not necessarily be packaged together in a kit.

FIG. 3B shows the gel layer 302 attached to surgical drape 304 with at least one piece of two sided adhesive tape 310. While the gel layer 302 is shown near the head of the patient, those skilled in the art will readily appreciate that the gel layer 302 can be attached to surgical drape 304 in any place along surgical drape 304 that is most convenient for the surgical procedure.

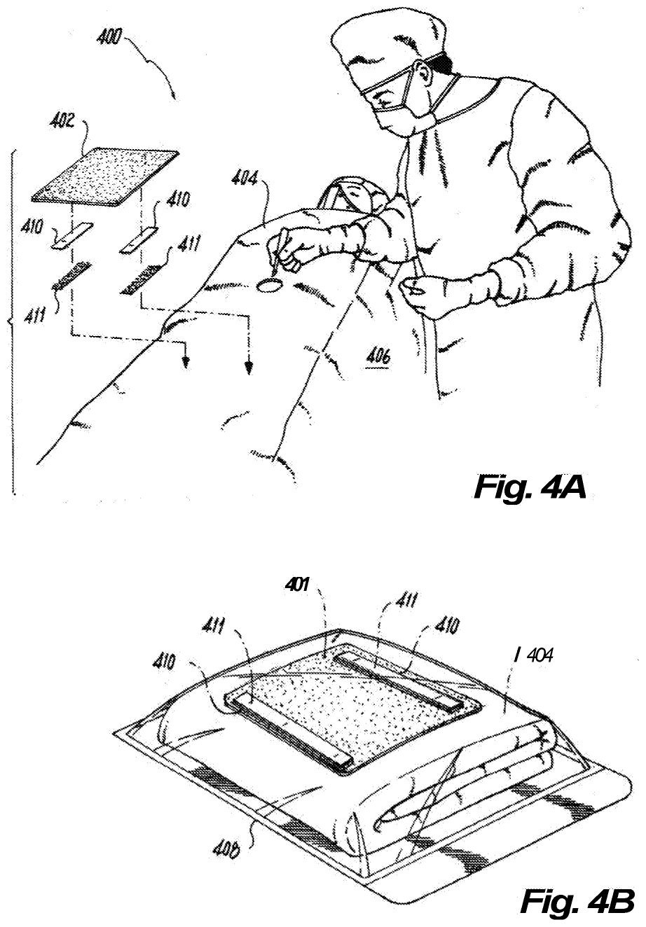

With reference now to FIG. 4A, at least one piece of hook and loop fastener 410 is affixed to one side of the gel layer 402 for attaching to surgical drape 404. One portion of the hook and loop fastener 410 is attached to gel layer 402 while the other portion of hook and loop fastener 411 can be sewn into surgical drape 404 or can be freely existing in sterile package 408 to be attached anywhere along surgical drape 404. It is contemplated that the gel 402 and attached hook and loop fastener 410 can be sterilized and enclosed in a sterile package 408 to maintain the sterile state until needed for use in a surgery. Those skilled in the art would readily appreciate that hook and loop fastener 410 and 411 and surgical drape 404 need not necessarily be packaged together in a kit.

FIG. 4B shows the gel layer 402 attached to surgical drape 404 with at least one piece of hook and loop fastener 410. Those skilled in the art will readily appreciate that the gel layer 402 can be attached to surgical drape 404 in any place along surgical drape 404 that is most convenient for the surgical procedure.

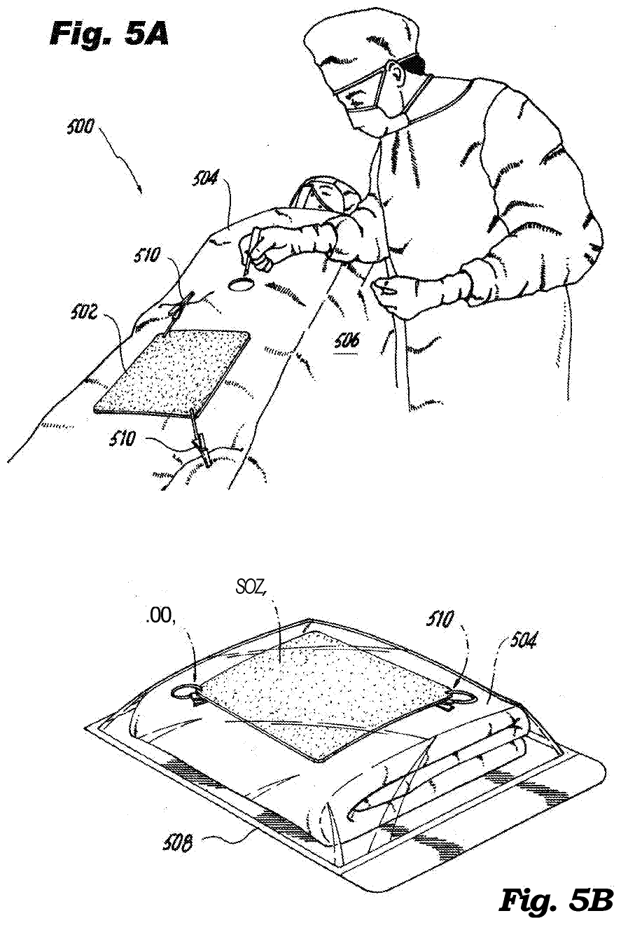

With reference now to FIG. 5A and 5B, at least one alligator clip 510 is affixed to one side of the gel layer 502 for attaching gel layer 502 to surgical drape 504. It is contemplated that the gel 502 and attached alligator clip can be sterilized and enclosed in a sterile package 508 to maintain the sterile state until needed for use in a surgery. Those skilled in the art would readily appreciate that alligator clips 510 and surgical drape 504 need not necessarily be packaged together in a kit. Those skilled in the art will readily appreciate that the gel layer 502 can be attached to surgical drape 504 in any place along surgical drape 504 that is most convenient for the surgical procedure.

The methods and systems of the present invention, as described above and shown in the drawings, provide for improved position maintenance of parked surgical instruments in a sterile field during a surgical procedure. While the apparatus and methods of the subject invention have been shown and described with reference to preferred embodiments, those skilled in the art will readily appreciate that changes and/or modifications may be made thereto without departing from the spirit and scope of the subject invention.

What is claimed is:

1. A system for parking and maintaining surgical instruments within a sterile field comprising:
a layer of force attenuating polymer gel configured and adapted to be sterilized and attached to a structure within the sterile field.

2. A system as recited in claim 1 further comprising:
at least one or more of: a sterile surgical tray and a sterile surgical drape.

3. A system as recited in claim 1, wherein the layer force attenuating polymer gel attached as a tray liner to a surgical tray for parking surgical instruments within the sterile field.

4. A system as recited in claim 3, wherein the layer of force attenuating polymer gel and the surgical tray are sterilized and enclosed in a sterile package.

5. A system as recited in claim 1, wherein the layer of force attenuating polymer gel is sterilized and is sewn in to a surgical drape for parking surgical instruments within the sterile field.

6. A system as recited in claim 5, wherein the layer of force attenuating polymer gel and the surgical drape are sterilized and enclosed in a sterile package.

7. A system as recited in claim 1, wherein the layer of force attenuating polymer gel is attached to a surgical drape in a manner where the parked surgical instruments are maintained at an angle that is less than forty-five degrees relative to the horizontal plane.

8. A system as recited in claim 1, wherein at least one piece of two sided adhesive tape is attached on one side to the layer of force attenuating polymer gel for attaching to a surgical drape.

9. A system as recited in claim 8, wherein the layer of force attenuating polymer gel with attached adhesive tape are sterilized and enclosed in a sterile package.

10. A system as recited in claim 1, wherein at least one piece of a hook and loop fastener is attached on one side to the layer of force attenuating polymer gel for attaching to a surgical drape.

11. A system as recited in claim 10, wherein one portion of the hook and loop fastener is attached to the layer of force attenuating polymer gel and the opposite portion of the hook and loop fastener is attached to the surgical drape.

12. A system as recited in claim 10, wherein the portion of the hook and loop fastener attached to the surgical drape is sewn into the surgical drape.

13. A system as recited in claim 10, wherein the portion of the hook and loop fastener that can be attached to the surgical drape is free to be placed anywhere along the surgical drape.

14. A system as recited in claim 10, wherein the layer of force attenuating polymer gel with attached hook and loop fastener and surgical drape are sterilized and enclosed in a sterile package.

15. A system as recited in claim 1, wherein at least one alligator clip is attached to the layer of force attenuating polymer gel for attaching to a surgical drape.

16. A system as recited in claim 12, wherein the layer of force attenuating polymer gel with attached alligator clips are sterilized and enclosed in a sterile package.
